(19) United States
(12) Patent Application Publication
(10) Pub. No.: US 2003/0138434 A1
(43) Pub. Date: Jul. 24, 2003

(54) Agents for enhancing the immune response

(76) Inventors: Robert L. Campbell, Bahama, NC (US); John A. Mikszta, Durham, NC (US)

Correspondence Address: VENABLE, BAETJER, HOWARD AND CIVILETTI, LLP, P.O. BOX 34385, WASHINGTON, DC, 20043-9998, US

(21) Appl. No.: 10/142,966

(22) Filed: May 13, 2002

Related U.S. Application Data
(60) Provisional application No. 60/311,387, filed on Aug. 13, 2001.

(51) Int. Cl. A61K 39/00

(52) U.S. Cl. 424/184.1

(57) ABSTRACT

The invention relates to an immunogenic composition and methods of making and using the composition. The immunogenic composition contains a directing molecule, a stimulant and an immunogen. The stimulant and directing molecule are chemically distinct. The stimulant and immunogen are present in relative amounts to result in an improved immune response relative to that resulting from the immunogen and just one of the directing molecule or stimulant.

### BACKGROUND OF THE INVENTION

1. Field of the Invention

This invention relates to the field of immunology and particularly to the combination of different classes of adjuvants to aide the host in generating an improved in vivo antibody response to pathogens, mammalian antigens and other clinically significant targets.

2. Description of Related Art

Seventy-five years ago Ramon demonstrated that it was possible to augment the antitoxin response to diphtheria and tetanus by administering vaccines with pyrogenic bacteria. Since that time, there has been an ongoing effort by clinicians and immunologists to enhance the immune response via adjuvants while attempting to minimize the often-present toxic side effects that prevent their use in humans.

In general antigens are “presented” to the immune system by antigen presenting cells, which include B-Cells, dendritic cells and macrophages in the context of major histocompatibility complex molecules (MHCs) on the APC surface. Normally, synthetic and natural antigens supplied as immunogens are thought to be taken up and partially digested by APCs, allowing smaller pieces of the original intact antigen to be expressed on the cell surface.

It is presently understood that T-lymphocytes versus B-lymphocytes, are relatively unable to interact with soluble antigen. Typically, T-lymphocytes require antigen to be processed and then expressed on the cell surface of APCs in the context of MHC molecules as noted above. T-Cells and specifically T-Cell receptors recognize antigen in the form of a bimolecular ligand composed of the processed antigen and one or more MHC molecules. It is believed that APCs must be activated to express co-stimulatory molecules before effective priming of T-Cells can occur.

Expanding on Denritic cells, DCs are thought to be the most potent antigen-presenting cells and apparently the only ones that can activate native (previously unstimulated) T cells in a primary immune response (Banchereau and Steinman 1998 Nature 392: 245-252). Activation of naive T cells is necessary if a vaccine is to produce full T cell immunity and optimal antibody responses. Unfortunately dendritic cells are rare. They comprise only 1 in 400 cells in the secondary lymphoid organs, 1 in 500 of WBCs and 1 in 1000 cells in most non-lymphoid tissue. The scarcity is compounded by the low frequency of naive T cells able to respond to any individual antigen epitope, or MHC-peptide complex, estimated to be as low as 1 in 10,000 (Mason 1998 Immunol. Today 19:395-404), In short, generating the best immune response comes down to the antigen reaching one rare cell that must subsequently interact with another rare immune cell.

Naïve T cells continuously recirculate through lymph nodes via the blood stream (Gretz et al 1996 J. Immunol. 157:495-499), whereas immature dendritic cells are relatively stable residents of non-lymphoid organs (Cowing and Gilmore 1992 J. Immunol. 148: 1072-1079). Immature dendritic cells express low levels of surface MHC and co-stimulatory molecules, as such are only weak cellular activators of T cells. However these cells are pinocytic and phagocytic, enabling them to constantly sample their environments for the presence of potential pathogens. When exposed to the appropriate “stimuli” (stimulant adjuvants), immature dendritic cells are mobilized, disengage from local tissue and migrate via the different lymphatics to drain in lymph nodes (Bancherau and Steinman 1998 Nature 392:245-252). During their migration to lymph nodes the DCs mature and become potent T cell activators. Langerhans cells are possibly the most studied of the DCs. They reside in the epidermal layer of the skin and mucous membranes where they are present in higher frequency than the immature dendritic cells found in other non-lymphoid organs. The ability to tailor adjuvants to each type of immune cell and specifically Langahern type DCs could have big payoffs.

For the reasons given above, many immunogens and portions of, epitopes, including both foreign and endogenous often go unrecognized or only weakly recognized by the immune system. It has been difficult and in some instances impossible to raise antibodies against such targets. This problem is often encountered when working with small haptens or developing subunit vaccines consisting of small non-immunogenic peptides. Traditional methods for preparing such vaccines present these antigens as macromolecules by conjugating to protein carriers, which are more immunogenic and are vaccine targets as well (combination vaccines). But too often the conjugate still fails to induce the cytotoxic T-lymphocyte (CTL) response to the lesser immunogenic material. In these instances an adjuvant is required. Unfortunately, many of current adjuvants (approved and in trials) may enhance some aspect of the immune response, but all too commonly fail to elicit the necessary level of CTL response, serum response and/or display some type of toxicity. The current opinion on viral infection, suggests protection is best accomplished by both humoral and cell-mediated immunities, including long-term memory and cytotoxic T-Cells.

US 2001/0024649 A1 describes topical administration of immunogen with some agents categorized in this invention as stimulants. The inventors provide an excellent summary of DC maturation and the need for stimulants however no mention of the combination of directing molecules and stimulants.

WO 99/43350 describes topical pretreatment agents and adjuvants, some of which this invention categorizes as stimulants, but does not discuss antigen targeting. Delivery routes are limited to IM, oral, nasal, and rectal.

In WO 02/11669 the inventors describe the combination of saponin with alpha 2-M and HSP. The document lists other response modifiers, which this invention categorizes as stimulants. While no data was provided nor evidence of a synergistic relationship, the combination of a single stimulant (saponin) with two different directing molecules was described. Since both directing molecules were to the same receptor (CD 91) the inventors failed to realize and demonstrate the broader relationship between the two classes of adjuvants. Further, since HSPs are associated with stressed cells and are expected to possess “stimulant” type-epitopes, the contribution from saponin is not clear. The inventors made no comment about the use of antibodies.

WO 99/13915 discusses antigen targeting, however falls short of describing the cooperation, which can be achieved when adjuvants of the two classes are combined. The work focuses on DNA antigens and implies the antigen and directing molecule must be conjugated with reagents such as PEI. Only a 2× enhancement was observed by a first delivery route, which appeared to be matched by delivering an antigen without the directing molecule by a second delivery route. As discussed earlier in this application, DNA antigens like traditional immunogens still require a stimulant. The WO document lists other directing molecules, some of which are categorized in this invention. US Published Application 20020022034 speaks to a method of delivering a gene delivery complex with targeting molecules along with an antiretroviral drug therapy.

From another perspective, there is also a need to reduce the toxicity animals are exposed to, when researchers and reagent developers attempt to make antibodies for in vivo and in vitro applications. In 1985, the U.S. Public Health service established guidelines mandating animal facilities to establish Institutional Animal Care and Use Committees (IACUC). The IACUCs are locally responsible for assuring the reduction of toxic agents such as Complete Freund's Adjuvant in animal experimentation. Until now, there has not been a substitute for CFA's potency without showing some level of toxicity, either local or systemic.

Historically, directing molecules and stimulants have been used separately because immunologists believed one class of adjuvants could compromise the other. In fact many of the antigen direction approaches were specifically intended to circumvent the need for the stimulant, and stimulants such as monophosphoryl lipid A (MPL) were once touted to be an independent option to Complete Freunds. Within this invention, immunogens and stimulants are intentionally employed with directing (presenting) antibodies and/or alpha 2-macroglobulin.

There is a need for adjuvants and methods for selecting such adjuvants, which promote; the capture of antigen by rare immature antigen presenting cells (APCs), the maturation and loading of APCs and finally an increase in APC interaction with antigen-specific T cells. These adjuvant attributes are needed for traditional immunogens as well as the newer DNA/RNA immunogens (Iwasaki et al 1997. J. Imminol. 159:11) which are subject to the same constraints. Antigens combined with an adjuvant filling only one role will not realize the best possible immune response.

There is a need for adjuvants that rival and ideally surpass the adjuvancy of Complete Freund's (CFA) without the toxicity associated with CFA. There is a need for new adjuvant formulas compatible with a large range of delivery routes and microdevice-based devices. New adjuvants are needed to work with less immunogen and targets that are poorly immunogenic, not require any processing beyond “add and mix”, and able to tolerate freezing and possibly lyophilizing without loss in activity. Accordingly the work that follows will intentionally avoid depots such as liposomes and oils that can also limit the effect of adjuvant cocktails, which are receptor-mediated.

### SUMMARY OF THE INVENTION

The invention relates to an immunogenic composition and methods of making and using the composition.

The immunogenic composition contains a directing molecule, a stimulant and an immunogen. The stimulant and directing molecule are chemically distinct. The stimulant and immunogen are present in relative amounts to result in an improved immune response relative to that resulting from the immunogen and just one of the directing molecule or stimulant.

The invention further relates to immunogenic compositions free from agents causing visible external toxic or allergic symptoms. Preferably the composition is alum-free. There is no requirement that the components be encapsulated or that liposomes be employed. The composition may be stored in a frozen or lyophilized state. It is intended that the first adjuvant and the second adjuvant be intermixed. The composition may include the addition of antibody or fragment, which is not specific for the immunogen, if desired. More clearly the composition may include the addition of antibody or fragment whereby the complimentarily determining regions (CDRS) are specific for immunogen, specific for APC and/or specific for stimulant. In constrast, it is not necessary that the CDR's be specific for APC, stimulant, or immunogen.

The invention further relates to compositions to induce the production of antibodies. The antibodies are suitable for diagnostic, research and therapeutic use. The composition can be used as a vaccine. Any pharmaceutically acceptable carrier may be added to the composition in this regard. The composition maybe administered to the subject employing any conventional mode of administration, including mucosal.

### BRIEF DESCRIPTION OF THE DRAWINGS

FIG. 1 shows activity stimulated by a composition of the invention at the 1:5,000 dilution without any optimization, such as varying antibodies, antigen: antibody ratios or immunization route.

FIG. 2 shows a pre-bleed with no activity toward the coated HCG antigen. HCG alone could not be called positive at the 1:3,333 dilution employing the 3× background criteria. The combination of HCG+saponin was an improvement over HCG alone. The combination of HCG and Alpha 2-Macroglobulin led to further improvement but the standout was clearly the combination of HCG (Immunogen)+Alpha 2-Macroglobulin (Directing Molecule)+Saponin (stimulant). The inventive combination also surpassed the HCG and Freunds Adjuvant combination (Data not shown).

FIG. 3 shows the combination of HCG and directing molecule (alpha 2-M) could not generate activity (3× background) at the lowest dilution assayed. The combination of HCG+stimulant (Qiagen's CpG) showed activity but only extending to the 1:270 dilution. In contrast, the combination of HCG with directing molecule (alpha 2-M) and stimulant (CpG) produced a functional titer adequate for monoclonal antibody applications.

FIG. 4 shows a representative test bleed from an animal that did not receive pancreatic membrane antigen (non-immune) with no activity at any dilution. A pool of serum from mice immunized with pancreatic membrane antigen and CpG was barely distinguishable from the non-immune at any dilution. In contrast, the combination of pancreatic membrane antigen with directing molecule (CK19 antibody) stimulant (CpG) produced a titer of 1:30 K, minimally a 250× enhancement over the inoculum containing CpG and Pancreatic antigen. Note PCMA and PMA are the same material.

FIG. 5 shows a representative test bleed from an animal that did not receive the inactivated chlamydia antigen (non-immune) with no activity at any dilution. Using a cutoff criteria of 2× bkgd+≧0.3OD a pool of serum from animals primed with chlamydia and CFA titered to 10K. In contrast a pool of serum from animals immunized with an adjuvant comprised of chlamydia EBs, L1 AB and saponin titered to 90K. A 9× enhancement.

FIG. 6 shows individual tissue fluid responses for each animal at a 1:8 screening dilution. Three animals did not receive any immunogen (non-immune 1-3) and served as negative controls. None of five animals receiving just chlamydia EB particles produced a positive signal (2× bkgd+≧0.3 OD). All five animals receiving chlamydia EB particles and saponin were negative. All 5 animals receiving chlamydia EB particles and L1 antibody were also negative. In contrast 3 of 5 animals receiving an inoculum comprised of chlamydia EB particles, L1 AB and saponin produced convincing specific IgA signals.

FIG. 7 shows individual tissue fluid responses for each animal at a 1:8 dilution. Three animals did not receive any immunogen (non-immunel-3) and served as negative controls. All five animals receiving inoculum comprised of chlamydia EB particles, L1 AB and saponin produced convincing specific IgA signals (2× bkgd+>0.3 OD), as did all five animals receiving inoculum comprised of chlamydia EB particles, alpha 2-macroglobulin and saponin.

### DETAILED DESCRIPTION OF THE INVENTION

As viewed by the inventors, “Adjuvant” is a broad term that seems to capture at least three categories of materials as classified by their function. One category of materials is those that function as depots. Examples of depots include Alum and Incomplete Freunds, which keep immunogen concentrated and control release. Another category is stimulants whereby surface antigens from organisms such as C. Parvum and plant extracts excite the antigen presenting cells and ultimately the broader immune response. The third category is immunogen directing or antigen targeting molecules that help to concentrate antigens on the surface of immune antigen presenting cells (APCs) and thereby enhancing uptake. Examples of this third type of substance are molecules such as antibodies and alpha 2-macroglobulin. While all adjuvants have a recognizable primary function, some possess a less obvious secondary trait. For example some oils will primarily operate as depots and to a smaller extent function as stimulants. Terms such as weak, mild and strong have often been used to describe adjuvant potency which historically correlated with toxicity.

Immunogen Selection

The immunogen can be any natural or synthetic antigen associated with or derived from any pathogen, cancer and other clinically significant target. More preferred antigenic materials include whole cells, proteins, carbohydrates, lipids or DNA. Whole cell “antigen” can include chlamydia, in particular trachomatis, pneumoniae or psittaci. Multiple types of immunogens can be employed where appropriate or desired, e.g. multivalent vaccines.

Five diverse immunogens were selected to demonstrate the synergistic responses that can occur when these two classes of adjuvants are appropriately paired. A purified protein preparation of human chorionic gonadotropin (HCG) was combined with alpha 2-M. The whole cell immunogen, chlamydia trachomatis elementary bodies was inactivated by three separate methods an matched with specific and non-specific murine antibodies to demonstrate the impact certain treatments can have on the selection of directing molecules. Further, an inoculum comprised of chlamydia, saponin and non-specific antibody was prepared and delivered by three separate routes to show the variation in performance, which can occur with different tissue. Finally, a mixture of mammalian membrane proteins was extracted from human Islet progenitor cells and combined with a commercially available antibody to demonstrate how an existing antibody can be leveraged to generate novel antibodies with similar and different properties.

Antigen Targetting and Stimulants

Antigen targeting became a common approach to avoiding the toxic effects experienced with the strongest adjuvants such as Complete Freunds and TiterMax of which neither are approved for human applications. Most of the work began in the late 1970's and was focused on enhancing the monoclonal antibody technology introduced by Kohler and Milstein in 1975. While responses were enhanced and toxicity eliminated, the overall increase to titers were marginal. Surprisingly, then and since, the efforts of many labs were focused on the independent use of directing molecules without consideration for the synergistic effects that may result if directing molecules were combined with certain “weaker adjuvants” showing none or limited symptoms of toxicity.

Many of the “weaker adjuvants” can be categorized as stimulants and do not cause persisting necroticing dermatitis or palpable lumps or ulceration of the skin as has been extensively documented for CFA. Used alone these weaker adjuvants found limited use. Immunologists typically resorted to combining such adjuvants with oils or incorporation into liposomes to increase potency. Unfortunately, these cocktails require more processing, can rarely be frozen or lyophilized (as needed for broad commercialization), are compatible with fewer delivery routes/devices and in some instances the toxicity is elevated as the adjuvant cocktail becomes more complex.

The capacity for antigen uptake by different APC appears to correlate with efficiency of presentation (Stockinger, B. 1992) and may involve antigen focusing or intracellular signaling. In general, targeting of antigen to the APC surface appears to enhance the immune response. The targeting of antigen to APC has been extensively studied in vitro and in vivo. For a review of antigen targeting see Fossum, S., et al., 1992. “Targeting Antigens to Antigen Presenting Cells”. Semin. In Immunol. 4:275 and more recent (Chattergoon M. A. et al., 2000) improved antigen presentation via targeting antigens directly to dendritic cells by capitalizing on the apoptosis cascade.

The stimulant is selected from conventional adjuvants having the characteristic, e.g. CpG DNA, nucleic acid. saponin, saponin derivatives, or saponin components having the requisite activity. Saponin derivatives include compounds, e.g. salts, having the saponin structure and stimulating activity. Saponin components are those saponin moieties having the same stimulatory activity of the natural compound, even though the activity may vary in degree. The saponins may be synthetic.

Saponins and particularly triterepene glycoside saponins are naturally occurring substances that can be harvested from Quillaja saponaria. Their adjuvant properties are well documented which include induction of CTL responses, stimulation of strong responses to T-dependent and T-independent antigens (Kensil R. C., 1996).

Saponin was selected from the stimulant class of adjuvants because of the range of responses it can excite and the relatively low levels of toxicity that are observed with the crudest of extracts. While this work was conducted with a purified saponin, more defined preps and synthetic versions, with increased performance are making their way through clinical trials, such as Stimulon QS-21. CpG is short sequences of DNA (oligonucleotides) that contain unmethylated cytosine-guanine dinucleotides within a certain base context. The mammalian immune system has evolved to recognize these sequences, which are found naturally in bacterial DNA, as an indicator of infection.

While the stimulant examples within are the glycoside; saponin and the nucleic acid; CpG, other types of molecules can be expected to work in a similar manner with a directing molecule. Specifically. GM-CSF, IL-IB, IL-2, IL-4, IL-7, IL-12, monophosphoryl lipid A (MPL), 3-Q-desacyl-4′-monophosphoryl lipid A (3D-MLA), formylated-met-leu-phe (fMLP) and IL-1beta 163-171 peptide (“Sclavo Peptide”). All of the priors have been described as “stimuli”, “stimulatory”, “immunostimulating” “stimulates” or “stimulant”. Further 25-dihydroxyvitamin D3 (calcitrol), calcitinin-gene regulated peptides, Dehydroepiandrosterone (DHEA), N-Acetylglucosaminyl-(P1-4)-N-acetylmuramyl-L-alanyl-D-glutamine (GMDP)/dimethyl dioctadecyla or disteary ammonium bromide (DDA)/Zinc L-proline, muramyl dipeptide (MDP), N-Acetylglucosaminyl-(P1-4)-N-acetylmuramyl-L-alanyl-D-glutamine (GMDP), N-acetyl muramyl-L-threonyl-D-isoglutamine (Threonyl-MDP), N-acetyl-L-alanyl-D-isoglutaminyl-L-alanine-2-(1,2-dipalmitoyl-sn-glycero-3-(hydroxy-phosphoryloxy) ethylamide monosodium salt (MTP-PE), Nac-Mur-L-Ala-D-Gln-OCH3, Nac-Mur-L-Thr-D-isoGln-sn-glycerol dipalmitoyl, Nac-Mur-D-Ala-D-isoGIn-sn-glycerol dipalmitoyl, 1-(2-methypropyl)-1H-imidazo[4,5-c]quinolin-4-amine, 4-Amino-otec-dimethyl-2-ethoxymethyl-1H-imidazo[4,5-c]quinoline-1-ethanol, N-acetylglucosaminyl-N-acetyhnuramyl-L-Ala-D-isoGlu-L-Ala-glycerol dipalmitate (DTP-GDP), N-acetylglucosaminyl-N-acetylinuramyl-L-Ala-D-isoGlu-L-Ala-dip almitoxy propylamide (DTP-DPP), gamma interferon, 7-allyl-8-oxoguano sine, Poly-adenylic acid-poly-uridylic acid complex, MIP-1a, MIP-3a, RANTES and dibutyl phthahate and dibutyl phthalate analogues and C5a are expected to work cooperatively with directing molecules. While the above are a diverse group of compounds each has adjuvant properties (alone) and is soluble in water. In a few cases the stimulants above will spontaneously form aggregates or micelles in aqueous solution and can be used to formulate liposomes. However, the concentrations used in this art are always below the critical micelle concentration (CMC), as was the case for the saponin examples that follow. Processing to achieve encapsulation was not pursued because of the restrictions described earlier for depots. C5a complement and the fragments are anticipated but are not preferred since complement proteins have been shown to possess opsonizing properties like antibodies. As discussed below, natural substances possessing both targeting and stimulus properties can compete with materials which specialize in one role. Having unique molecules for each role will afford the most tuning. Evidence to support this rational is within, as unprecedented serum Ig enhancements of 250× over potent established adjuvants is demonstrated.

The stimulants described within are characterized by their relationship to humans whereby CpG and saponins are examples of exogenous stimulants. IL-2 and GM-CSF are examples of endogenous stimulants, part of the immune cascade and are produced in humans.

Saponin and CpG were selected for these studies because each has good solubility, they are vastly different molecules and thereby with success demonstrating the broad relationship, which exists between stimulants and targeting molecules when appropriately paired.

The invention further defines stimulants as substances contributing to APC disengagement from tissue, APC migration toward lymph nodes and APC maturation. The stimulants are often cytokines and more particularly chemokines or having chemoattractant properties.

Antigen Targeting With Antibodies

Each directing molecule and its corresponding receptor on an antigen-presenting cell can be expected to bind with different efficiency and subsequently facilitate the immune cascade at different rates. Some directing molecules have receptors on multiple types of APCs where the receptor frequency on each type of cell is expected to be unique. As discussed earlier the type of presenting cells and number can vary with tissue type. Also, the interaction between each immunogen and directing molecule will be distinct. Accordingly, the selection of a directing molecule should be a careful one, as the ability to facilitate uptake will vary with immunogen and administration route. Likewise for the selection of stimulant.

A long list of publications on antigen targeting with antibodies exists. The previous antibody work includes both natural and synthetic tethering strategies such as cross-linking antigen directed against surface proteins (Kawamura and Berzofsky, 1986) and forming immune complexes for recognition by FcR (Manca et al., 1991). Immunogens modeled in the earlier studies included Strep M glycopeptides (Bahr et al., 1985) where antibody to an adjuvant component (anti-muramyl dipeptide) was used, Hepatitis antigen (Balsap et al., 2000) where antigen specific IgM was involved and a Malaria antigen (Harte et al., 1983) also combined with a specific IgM. Where the earliest antigen targeting work was focused on generating monoclonal antibody (MAB) reagents, the current ability to humanize proteins and particularly antibodies brings the prospect of using antigen targeting (via antibodies) for vaccines closer to reality. The following are FDA approved antibody therapeutics with various degrees of humanization:

Orthoclone (anti-CD3), ReoPro (anti-11b/11a), Rituxan (anti-CD20), Zenapax (anti-IL2 receptor), Herceptin (anti-Her2 receptor), Remicade (anti-TNF), Synagis (anti-RSV), Simulect (anti-IL2), Mvlotara (anti-CD33), Campath (anti CD52).

The compositions or formulations taught by the present invention can be employed for inducing antibody. The composition is administered to a subject at any desired site to induce antibody formation. The immunogen may be present in amounts as low as 40 ng/boost or even lower, e.g. about 8 ng/boost. If desired one or more of the components of the composition may be separately administered to the subject or if a component is otherwise present in the subject at the desired site, the composition may be modified so that the desired effect is achieved. The antibody induced has Ka values between 10⁴-10¹³ moles/liter and may be recovered from the subject. The recovered antibody is then analyzed for its affinity for the antigen epitopes. Further, protective titers of the antibody are achieved. The composition can be used as a vaccine. Any pharmaceutically acceptable carrier may be added to the composition in this regard. The composition may be administered to the subject employing any conventional mode of administration, including mucosal. Further administration can occur by more than one route and by more than one route either simultaneously or in sequence. A series of vaccinations is possible.

Antigen Targeting With Alpha 2 Macroglbulin

The directing molecules used within include alpha 2-macroglobulin (alpha 2-M or A2M) prepared by different methods and antibodies at different purities with various specificities. Complement and other oposonizing molecules are anticipated. Fragments of these molecules capable of binding immunogen are also envisioned as directing molecules. The directing molecule does not need to be specific for the immunogen present in the composition. The antibody examples within are specific to the immunogen as well as non-specific. The directing molecule can be specific for an APC receptor. Alternatively the targeting molecule may be directly or indirectly linked to an APC receptor. While antibodies and alpha 2-M are preferred directing molecules, those binding to transferrin, manose and asialoglycoproteins receptors are anticipated. Mammalian heat shock proteins are anticipated but are not preferred since it is well established that HSPs are stress proteins and may harbor “stimulating” epitopes. Natural substances possessing both targeting and stimulus properties can compete with materials which specialize in that role. As specified earlier, having unique molecules for each role will afford the most tuning.

While B cells possess specific receptors for Ig, (Rock et al., 1984), macrophages and other non-B Cell APCs are known to use other mechanisms, which include phagocytosis and endocytosis. The uptake and presentation of soluble antigen by macrophages is not fully understood. However, a case has been made for a receptor-mediated mechanism with alpha 2-macroglobulin and CD91 (Binder, R. J. et al., J. of Immunol, 2001, 166:4968-4972), the same receptor used by heat shock proteins.

Among the immune cells, macrophages are of particular interest due to the pivotal role they play in the broader immune system. The ability of macrophage to regulate a range of immunological interactions is due in part to their expression of la surface antigens. The expression of membrane la antigens is essential for the induction of specific T-Cell responses to antigens (Unanvel, 1981). For these reasons, the alpha 2-macroglobulin system is currently receiving a lot of attention and the enhancements with alpha 2-M are being compared with the earlier antigen targeting data generated with antibodies.

Human alpha 2-M is an abundant protein in plasma (2-5 mg/ml). It consists of four identical subunits arranged to form a double-sided molecular “trap”. The trap is triggered when enzyme or methylamine activate a highly susceptible stretch of amino acids, the “bait region,” leading to a traceable conventional change (Barret and Starkey, 1973). The resulting receptor-recognized alpha 2-M is efficiently internalized by macrophages, dendritic cells and other cells expressing alpha 2-M receptors (Pizzo et al., 1984). To date, binding alpha 2-M to non-proteolytic proteins, either naturally or synthetically does not appear to effect internalization by APCs, although size and charge may affect the extent of binding. Alpha 2-M will likely see broad use in future antigen-targeting applications.

Antibodies and alpha 2-M were selected for these studies because each is present in the systemic circulation and tissue in high concentrations under normal conditions and thereby would not be expected to possess stimulant type epitopes that would confound results. In addition, alpha 2-M and antibody structures are vastly different molecules and utilize different APC receptors, thereby with success demonstrating the broad relationship, which exists between stimulants, and targeting molecules when appropriately paired.

### EXAMPLES

An Antibody Directing Molecule, Whole Cell Immunogen (Chlamydia EB) and Saponin Stimulant

The three chlamydiae species infecting humans are Trachomatis, Pneumonia and Psittaci. Infections of the eye and genital tract by trachomatis are a major cause of morbidity worldwide and are costly to treat. Development of a vaccine capable of protecting against infection or severe disease presents special challenges but would be the most effective long-term option for control of chlamidea disease.

Pneumoniae (TWAR) was first isolated from respiratory infections approximately 15 years ago and has subsequently been shown to be a common cause of pneumonia, bronchitis, sinusitis and pharyngtis. More recently, the presence of pneumoniae in atheroma of the coronary artery has many pharmaceutical companies taking a traditional vaccine approach to treating atherosclerosis. Since the chlamydiae are serologically similar, it is possible that a single vaccine could provide protection against direct and indirect chlamydia disorders.

Chlamydiae have developed the ability to evade the immune system. Most believe that protection against these pathogens will require a strong humoral and cell-mediated response. Progress has been made in understanding the pathology of these infections and protection. The most promising data has been generated with whole cells, the major outer membrane protein (MOMP) and MOMP DNA. When selecting a vaccine approach, whole cell immunogens are generally more immunogeninc than subunit vaccines. This is also true for chlamydia; however, when the whole chlamydia particle is inactivated it has historically lost its immunogenicity. Example 1 shows how the immunogenicity of an inactivated chlamydia particle can be enhanced by combining with directing molecule and stimulant class adjuvants.

### Example 1

| | |
|---|---|
| Immunogen = | Whole Cell (Chlamydia Trachomatis) |
| Directing = | Antibody (Murine, specific to Chlamydia LPS) |
| Molecule | |
| Stimulant = | Saponin |

Immunogen Preparation

Approximately 10⁸ formalin fixed chlamydia trachomatis (L2 serovar) elementary bodies (EBs) were dispensed into 100 ul of HBSS. To the EB stock, 90 microgram of purified #403 murine antibody was added in 25 ul of phosphate buffered saline. Finally, 2 ul being 10 ug of purified saponin was added. The above was mixed and allowed to incubate at room temp for 10 minutes. Afterwards the volume was brought to 1 ml allowing each animal to receive 200 ul of the prepared inoculum.

Immunization

Five balb/c mice were designated for traditional CFA immunization, 5 were designated for the novel adjuvant combination and each possible component combination as controls. All animals received four IP immunizations at 2×10⁷ each in 200 ul of Hanks Buffered Saline, which were spaced two weeks apart. The total inoculum was always 200 ul.

TABLE 1

| Balb/c Mice | Balb/c Mice | Balb/c Mice | Balb/c Mice | Balb/c Mice |
|---|---|---|---|---|
| Group 1 | Group 2 | Group 3 | Group 4 | Group 5 |
| Cntrl | Cntrl | Cntrl | Invention | Cntrl |
| Chlamydia | Chlamydia | Chlamydia | Chlamydia | Chlamydia |
| Immunogen + | Immunogen + | Immunogen + | Immunogen + | Immunogen + |
| Traditional | Stimulant | Directing | Directing | Non Specific |
| CFA/IFA | (Quil A | Molecule | Molecule | Antibody + |
| Adjuvant | Saponin) | (Specific | (Specific | Stimulant Quil |
| | | Antibody) + | Antibody) + | A Saponin) |
| | | Traditional | Stimulant | |
| | | CFA/IFA | (Quil A | |
| | | Adjuvant | Saponin) | |
| Day 0 | Day 0 | Day 0 | Day 0 | Day 0 |
| IP | IP | IP | IP | IP |
| Immunization | Immunization | Immunization | Immunization | Immunization |
| Day 13 | Day 13 | Day 13 | Day 13 | Day 13 |
| IP Boost | IP Boost | IP Boost | IP Boost | IP Boost |
| Day 26 | Day 26 | Day 26 | Day 26 | Day 26 |
| IP Boost | IP Boost | IP Boost | IP Boost | IP Boost |
| Day 39 | Day 39 | Day 39 | Day 39 | Day 39 |
| IP Boost | IP Boost | IP Boost | IP Boost | IP Boost |
| Day 40 | Day 40 | Day 40 | Day 40 | Day 40 |
| Test Bleed | Test Bleed | Test Bleed | Test Bleed | Test Bleed |

A test bleed was taken after the fourth IP injection and titered vs. CT L2 infected MeCoy Cells in a solid phase ELISA.

Methods and Materials Used to Quantify Levels of Antibody to Chlamydia Immunogen

Materials: PVC plates, L2 Chlamydia Trachomatis (CT) MeCoy Lysate, bovine serum albumin, phosphate buffered saline (PBS), carbonate buffer, phosphate buffered saline with 0.5% tween 20 (PBS-T), DMEM media with 20% serum, 5 ml glass tubes, pools of test bleeds taken from each cage on day 40, Goat anti-mouse IgG (whole molecule) HRP Sigma A-4416, phosphate-citrate buffer capsule Sigma P-4922, OPD substrate 30 mg tablet Sigma P-8412, 4.5 molar sulfuric acid

ELISA Procedure:

1. Coat PVC plates at 10⁶ chlamydia trachomatis particles/ml with 100 μl per well pH 9.5 carbonate buffer by 1 hour at 37 degrees C.

2 Dump coat and block for 1 hour by flooding wells with a 3% BSA in PBS and incubating at 37 degrees C.

3 Dump block and rinse 2× with PBS-T by flooding wells.

4. Add 50 ul of each test/pre-bleed pool dilution and incubate for 1 hour at 37 degrees C. Each dilution is made with a solution that is 90%PBS-T and 10% DMEM media that is 20% serum.

5. Dump and rinse 3× by flooding wells each time.

6. Add 50 ul of a Goat anti-mouse IgG HRP diluted at 1:1 OK with PBS-T and incubate at 37 degrees C. for 1 hour.

7. Dump and rinse 3× by flooding wells each time.

8. Add 50 ul of a citrate-phosphate buffer that is 2 mg/ml OPD and incubate until the lowest dilution of the highest response is estimated to reach 1 OD when scanning at 495 nm.

9. Stop the reaction with 50 ul of 4.5 molar sulfuric acid and read at 495 nm with a Titertek plate reader for final record.

Summary of Chlamydia Results

In a series of experiments one stimulant (purified saponin “Quil A”), when mixed with inactivated Chlamydia Trachomatis (CT) particles complexed with C-LPS specific antibody was found to produce an immediate and sustained increase in serum titer. The result was particularly surprising since the enhancement was over Complete Freunds Adjuvant and not just an enhancement over immunogen alone.

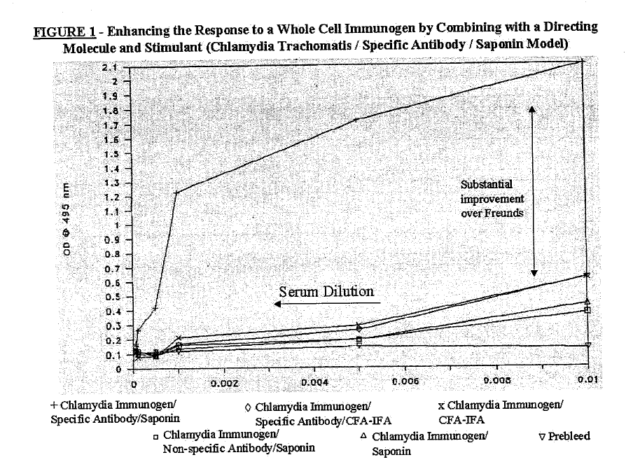

The published literature shows mice (post immunization) and humans (post infection) with titers to chlamydiae of approximately 1:500. The titers stimulated by the novel combination described above and in FIG. 1 were tracking at 1:5,000 or beyond without any optimization, such as varying antibodies, antigen: antibody ratios or immunization route.

Dilutions were started at 1:100 and compared against a prebleed and a representative CFA response. The prebleed was negative for CT antibody at 1:100 and the CFA treatment could only be called positive at the lowest dilution by dropping the qualifying value to 30% of max signal. Note, the specific antibody and immunogen combination did not show an improvement over Freunds. Likewise, the Quil-A and immunogen combination did not show an improvement over Freunds.

An Alpha 2-Macroglobulin Directing Molecule, Purified Protein Immunogen (HCG) and Saponin Stimulant

HCG is a glycoprotein with a molecular weight of 38,000 Daltons. It is composed of 2 subunits, the alpha and beta chain. The alpha subunit consists of a 92-aa sequence, which is identical to the pituitary glycoproteins: FSH, LH AND TSH. The beta subunit, the N-terminus 115-aa piece is the same as the beta-LH subunit; however, the C-terminal 30-aa sequence is unique and often referred to as the business end of the molecule. HCG is secreted by the placenta and levels increase during the first trimester of pregnancy.

HCG is an excellent candidate for evaluating any enhancement that may come with an HCG-A2M immunogen. Many academic and diagnostic labs over the last two decades have tracked titers to HCG on their way to generating reagents for OTC pregnancy tests and thereby creating a lot of serum titer data for comparison.

Alpha 2-macroglobulin may provide another benefit. Once antigen is internalized by APCs, partial proteolytic degradation occurs in an endosome and processed peptide fragments of the antigen become associated with MHC molecules. However, while partial proteolytic degradation of the antigen may be essential to generating appropriate MHC and T-Cell binding to the peptide fragments thereof, excessive degradation may prove detrimental to the immune response. Complete proteolysis is not essential for processing and an alpha 2-M mask may protect key epitopes needed for neutralization and protection.

Until now it was not proven that alpha 2-M could contribute to generating antibodies with the necessary Ka values (ranging from 10⁴-10¹³ moles/liter) for diagnostic applications while avoiding the toxicity typically imposed on laboratory animals. Further, with the disclosed adjuvant formulations, immunogens given at low concentrations can stimulate functional antibody titers. The term “functional titer” has different meanings in each field. A functional titer for those that practice the monoclonal antibody tools developed by Kohler and Milstein is generally 1:10,000. Animals with titers less than 10,000 are typically not good sources for primed B-Cells intended for fusion. A functional titer may also be described by the quality of antibodies within, such as containing neutralizing or protective antibodies.

### Example 2

| | |
|---|---|
| Immunogen = | Purified Protein (HCG) |
| Directing Molecule = | Macroglobulin (Murine, Alpha 2) |
| Stimulant = | Saponin |

Immunogen Preparation

The HCG was obtained from BioPacific of 4240 Hollis Street, Emeryville Calif. 94608 and was provided as a lyophilized powder from a 50 mM ammonium carbonate solution.

Several laboratories have experience with conjugating substances to alpha 2-M. Linkers for general protein-protein coupling may also be used and are available from Pierce Chemicals with easy to follow instructions. Synergy Vaccines Inc. is an “Incubator Company” located at Becton Dickinson's RTP, NC facility and was selected to carry out the conjugation step.

Immunization and Serum Sampling

All immunizations were given intraperitoneal (IP) at two-week intervals. Again, the IP route was selected thereby creating an option for later harvesting of splenetic B-Cells and generation of MABs for individual antibody analysis. Each animal received 1 ug of HCG per boost in 200 ul of Hanks Buffered Saline. Serum samples were taken from each animal in a test group and pooled for titration.

TABLE 2

| Balb/c Mice | Balb/c Mice | Balb/c Mice | Balb/c Mice | Balb/c Mice |
|---|---|---|---|---|
| Group 1 | Group 2 | Group 3 | Group 4 | Group 5 |
| Invention | Cntrl | Cntrl | Cntrl | Cntrl |
| HCG + | HCG ± | HCG + | HCG + | HCG |
| Alpha 2 | Saponin | Freunds | Alpha 2 | |
| Macro- | | Complete/ | Macro- | |
| globulin + | | Incomplete | globulin | |
| Saponin | | | | |
| Day 0 | Day 0 | Day 0 | Day 0 | Day 0 |
| IP | IP | IP | IP | IP |
| Immuni- | Immuni- | Immuni- | Immuni- | Immuni- |
| zation | zation | zation | zation | zation |
| Day 14 | Day 14 | Day 14 | Day 14 | Day 14 |
| IP Boost | IP Boost | IP Boost | IP Boost | IP Boost |
| Day 27 | Day 27 | Day 27 | Day 27 | Day 27 |
| IP Boost | IP Boost | IP Boost | IP Boost | IP Boost |
| Day 33 | Day 33 | Day 33 | Day 33 | Day 33 |
| Test Bleed | Test Bleed | Test Bleed | Test Bleed | Test Bleed |

Methods and Materials Used to Quantity Serum Levels of Antibody to HCG

Materials: PVC plates, HCG, bovine serum albumin, phosphate buffeted saline (PBS), carbonate buffer, phosphate buffered saline with 0.5% tween 20 (PBS-T), DMEM media with 20% serum, 5 ml glass tubes, pools of test bleeds taken from each cage on day 33, Goat anti-mouse IgG (whole molecule) HRP Sigma A-4416, phosphate-citrate buffer capsule Sigma P-4922, OPD substrate 30 mg tablet Sigma P-8412, 4.5 molar sulfuric acid.

Procedure:

1. Coat PVC plates at 1 ug/ml conc with 100 ul of HCG in pH 9.5 carbonate buffer by 1 hour at 37 degrees C.

2. Dump coat and block for 1 hour by flooding wells with 3% BSA in PBS and incubating at 37 degrees C.

3. Dump block and rinse 2× with PBS-T by flooding wells.

4. Add 50 ul of each test/pre-bleed pool dilution and incubate for 1 hour at 37 degrees C. Each dilution is made with a solution that is 90%PBS-T and 10% DMEM media that is 20% serum.

5. Dump and rinse 3× by flooding wells each time.

6. Add 50 ul of a Goat anti-mouse IgG HRP diluted at 1:1 OK with PBS-T and incubate at 37 degrees C. for 1 hour.

7. Dump and rinse 3× by flooding wells each time.

8. Add 50 ul of a citrate-phosphate buffer that is 2 mg/ml OPD and incubate until the lowest dilution of the highest response is estimated to reach 1 OD when scanning at 492 nm.

9. Stop the reaction with 50 ul of 4.5 molar sulfuuric acid and read at 492 nm with a Titertek plate reader for final record.

Titer and Toxicity Testing

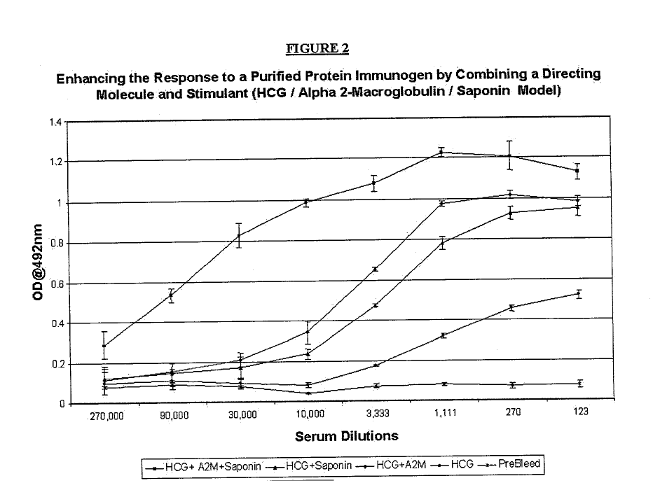

FIG. 2 shows a pre-bleed with no activity toward the coated HCG antigen. HCG alone could not be called positive at the 1:3,333 dilution employing the 3× background critieria. The combination of HCG+saponin was an improvement over HCG alone. The combination of HCG and Alpha 2-Macroglobulin led to further improvements but the standout was clearly the combination of HCG (Immunogen)+Alpha 2-Macroglobulin (Directing Molecule)+Saponin (stimulant). The inventive combination also surpassed the HCG and Freunds Adjuvant combination (Data not shown).

More importantly, the inoculum given to Groups 1 and 3 (above) were prepared again and delivered subcutaneously to a second group of five mice for the sole purpose of monitoring toxicity. Again, all animals received 200 ul of the inoculum, however, this time the material was divided amongst 6 sites. The animals were monitored for two weeks afterwards with the discerning results developing in the quadriceps and the footpad. Table 3 summarizes the observations.

TABLE 3

| | HCG + Freunds | HCG + Alpha 2- |
|---|---|---|
| | Complete/Incomplete | Macroglobulin + Saponin |
| Footpad | 5/5 Edema and erythema | Normal |
| Hind quadricep | 2/5 opened abscesses | Normal |

As shown in Table 3, the animals receiving HCG+Alpha 2-M+Saponin did not show any topical signs of toxicity versus the animals receiving HCG emulsified in Freund's which produced the well documented abscesses.

An Alpha 2-Macroglobulin Directing Molecule, Purified Protein Immunogen (HCG) and CpG Stimulant

### Example 3

| | |
|---|---|
| Immunogen = | Purified Protein (HCG) |
| Directing Molecule = | Macroglobulin (Murine, Alpha 2) |
| Stimulant = | CpG DNA |

Immunogen Preparation

The CpG stimulant used in this example was IumunoEasy™ purchased from Qiagen Inc. 28159 Stanford Avenue Valencia Calif. 91355. CpG stimulant is short pieces of DNA that contain unmethylated cytosine and guanine dinucleotides within a specific base content. Again, the HCG was provided by BioPacific and further processed in-house. The HCG+Alpha 2-M complex was prepared by Synergy Vaccines Inc. as indicated earlier.

Immunization and Serum Sampling

Since prior examples for creating functional titers were exclusively IP injections, this work only involved subcutaneous (SQ) immunizations and thereby demonstrating the invention was not limited to a particular route. Each animal received 40 ng of HCG weekly in a 200 ul volume. Hanks Buffered Saline was used when diluent was needed. Serum samples were taken from each animal in a test group and pooled for titration.

TABLE 4

| Balb/c Mice | Balb/c Mice | Balb/c Mice |
|---|---|---|
| Group 1 | Group 2 | Group 3 |
| Cntrl | Cntrl | Invention |
| HCG + CpG | HCG + Alpha 2-M | HCG + Alpha 2-M + |
| | | CpG |
| Day 0 | Day 0 | Day 0 |
| SQ Immunization | SQ Immunization | SQ Immunization |
| Day 7 | Day 7 | Day 7 |
| SQ Boost | SQ Boost | SQ Boost |
| Day 14 | Day 14 | Day 14 |
| SQ Boost | SQ Boost | SQ Boost |
| Day 20 | Day 20 | Day 20 |
| SQ Boost | SQ Boost | SQ Boost |
| Day 25 | Day 25 | Day 25 |
| SQ Boost | SQ Boost | SQ Boost |
| Day 27 | Day 27 | Day 27 |
| Test bleed | Test Bleed | Test Bleed |

Methods and Materials Used to Quantify Levels of Serum Antibody to HCG was Identical to Those Specified in Example 2.

Serum Titer Results

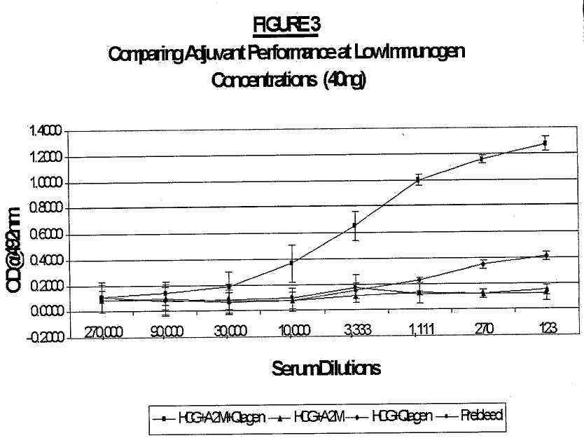

FIG. 3 shows the combination of HCG and directing molecule (alpha 2-M) could not generate activity (3× background) at the lowest dilution assayed. The combination of HCG+stimulant (Qiagen's CpG) showed activity but only extending to the 1:270 dilution. In contrast, the combination of HCG with directing molecule (alpha 2-M) and stimulant (CpG) produced a functional titer adequate for monoclonal antibody applications.

A Murine Antibody to Cytokeratin (Dir. Mol.) and CpG DNA Stimulant Used to Enhance the Response to Pancreatic Cell Membrane Antigen

Pancreatic cell membrane antigen (PCMA) is a complex of proteins collected from human pancreatic cells comprised primarily of ductal, and aciner type cells. Companies attempting to use stem or progenitor cells to treat diseases like diabetes are using these membrane proteins to monitor and direct the evolution of islet progenitors toward functioning insulin producing cells. These membrane protein markers, like many cancer markers are often transiently expressed and are only present in low concentrations during peak production. Being of human origin, transiently expressed and in low copy during peak expression has made it difficult, and often impossible to make antibodies to such targets. Accordingly, human PCMA is another excellent candidate for evaluating any enhancement that may come from combining stimulant and directing molecule type adjuvants. Any antibody produced from the immunizations will likely become a valuable research and/or development reagent for those working on islet cell therapies.

### Example 4

| | |
|---|---|
| Immunogen | Protein Complex (Pancreatic Cell Membrane |
| | Antigen) |
| Directing Molecule | Antibody (Murine, specific to CK19) |
| Stimulant | CpG |

Immunogen Preparation

The total immunogen prepared for test and control immunization schedules originated from a single 1502 cm flask (2 days after planting) of human pancreatic cells. Media containing serum was aspirated from the flask of adherent cells and the inside of the flask was rinsed 4× with HBSS before scraping cells from the surface. Harvested cells were placed in a 50 ml conical tube and centrifuged at 2,000 rpms for 5 minutes. Supernatant was discarded and 20 mls of fresh HBSS was used to resuspend cells. The process was repeated 3 times to remove any residual media constituents. After the fourth spin, the pellet was suspended in 10 mls of a pH 8 lysis buffer containing: 10 mM HEPES, 1 mM MgCl₂, 1 mM EDTA and 1 mM PMSF. The mixture was vortexed briefly and allowed to incubate on ice for 10 minutes. Afterwards the lysate was centrifuged for 10 minutes at 3,000 rpms. The supernatant was removed and centrifuged again at 100×g for 90 minutes. The supernatant was discarded and the pellet dissolved in a 1,300 ul of lysis buffer. The final stock concentration of 14.3 ug/ml was determined by a Lowry protein assay. The CpG stimulant used in this example was ImmunoEasy™ purchased from Qiagen Inc. The CK19 murine antibody was obtained from Biogenex Cat. AM246-5M and used in the whole ascites form.

Immunization

Five Balb/c mice received the immunogen plus CpG and five received the immunogen plus the novel adjuvant combination of CpG and CK19 antibody. Both groups received weekly IP immunizations over a 4-week period followed by a test bleed three days after the fifth boost. Beyond the test bleed, animals were immunized on day 40 and day 46. Spleens were removed for PEG fusions on day 49. Each animal in test and control groups received 20 ul or 286 ng total protein per boost. Polyacrylamide gel electrophoresis confirmed the PCMA was a mixture of at least 10 separate proteins or antigens of equal proportions suggesting each protein immunized was present at about 28 ngs. CpG animals received a mix containing 50 ul of Qiagen CpG, 150 ul of hanks buffered saline and 20 ul of the immunogen stock as described above. Animals receiving the inventive cocktail received 50 ul of Qiagen's CpG, 10 ul of Biogenex ascites (being between 10 and 100 ug of CK19 specific antibody), 120 ul of HBSS and 20 ul of the immunogen stock.

TABLE 5

| Control Group 1 Balb/c Mice | Test Group 2 Balb/c Mice |
|---|---|
| Pancreatic Cell Membrane | Pancreatic Cell Membrane Antigen + |
| Antigen + CpG | CpG + CK19 murine antibody |
| Day 0, IP Immunization | Day 0, IP Immunization |
| Day 7, IP Boost | Day 7, IP Boost |
| Day 13, IP Boost | Day 13, IP Boost |
| Day 21, IP Boost | Day 21, IP Boost1 |
| Day 28, IP Boost | Day 28, IP Boost |
| Day 31, Test Bleed | Day 31, Test Bleed |
| Day 40, IP Boost | Day 40, IP Boost |
| Day 46, IP Boost | Day 46, IP Boost |
| Day 49, Harvest Spleens | Day 49, Harvest Spleens |

The day 31 test bleed was titered using similar pancreatic cell membrane prep in a solid phase ELISA.

Methods and Materials Used to Quantify Levels of Serum Antibody to Pancreatic Cell Membrane Antigen

Materials: PVC plates, Pancreatic cell membrane antigen, bovine serum albumin, phosphate buffered saline (PBS), carbonate buffer, phosphate buffered saline with 0.5% tween 20 (PBS-T), DMEM media with 20% serum, 5 ml glass tubes, pools of test bleeds taken from each cage on day 31, Goat anti-mouse IgG (whole molecule) HRP Sigma A-4416, phosphate-citrate buffer capsule Sigma P-4922, OPD substrate 30 mg tablet Sigma P-8412, 4.5 molar sulfuric acid

ELISA Procedure:

1. Coat PVC plates with pancreatic cell membrane antigen at 3.34 ug/ml conc. with 100 μl per well pH 9.5 carbonate buffer by 1 hour at 37 degrees C.

2 Dump coat and block for 1 hour by flooding wells with a 3% BSA in PBS and incubating at 37 degrees C.

3 Dump block and rinse 2× with PBS-T by flooding wells.

4. Add 50 ul of each test/non-immune serum dilution and incubate for 1 hour at 37 degrees C. Each dilution is made with a solution that is 90%PBS-T and 10% DMEM media that is 20% serum.

5. Dump and rinse 3× by flooding wells each time.

6. Add 50 ul of a Goat anti-mouse IgG HRP diluted at 1:10K with PBS-T and incubate at 37 degrees C. for 1 hour.

7. Dump and rinse 3× by flooding wells each time.

8. Add 50 ul of a citrate-phosphate buffer that is 2 mg/ml OPD and incubate until the lowest dilution of the highest response is estimated to reach 1 OD when scanning at 495 nm.

9. Stop the reaction with 50 ul of 4.5 molar sulfuric acid and read at 495 nm with a Titertek plate reader for final record.

PEG Fusion and Recovery of MABS to Rare Targets

While the titer data did not justify pursuing MABs from the CpG schedule, spleen cells were collected from both CpG and CpG-Ck19 schedules. Approximately 2×10⁸ spleen cells were fused from each schedule. Ten days later plates (10) from each schedule were screened for the total hybrids and number of hybrids producing specific antibody.

TABLE 6

| | Pancreatic Cell Membrane | |
|---|---|---|
| | Antigen + CpG + CK19 | Pancreatic Cell Membrane |
| | Antibody | Antigen + CpG |
| Hybrids | 98 | 481 |
| Screened | | |
| Pos. Clones | 4 | 0 |

Serum Titer and Fusion Results

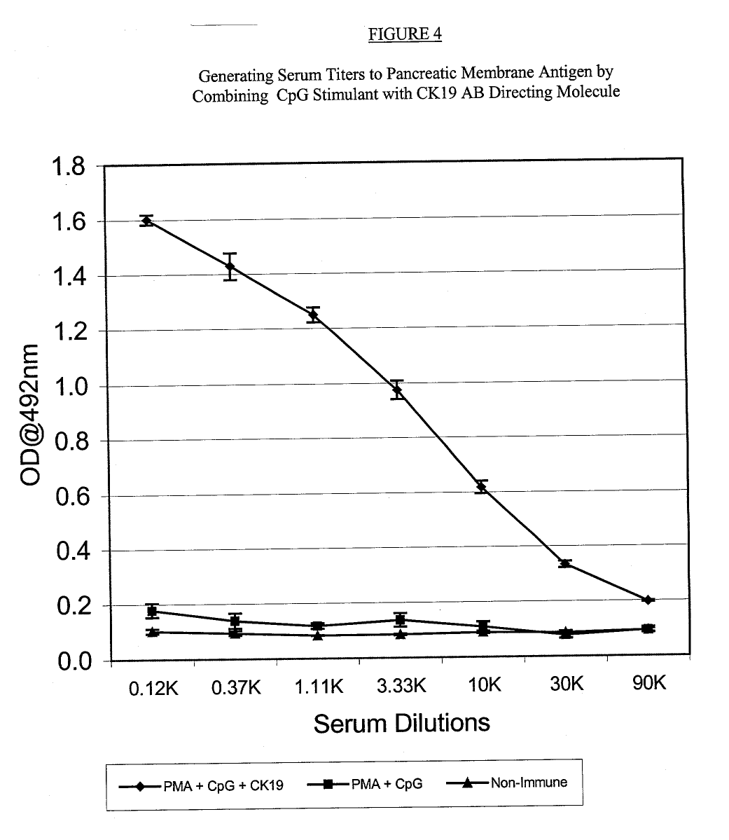

FIG. 4 shows the CpG stimulant could not generate activity (3× background) at the lowest dilution assayed. In contrast, the combination of directing molecule (murine CK19 antibody) and stimulant (CpG) led to a titer of 1:30K (3× Bkgd) easily satisfying in-house criteria (1:10K) for moving forward with efforts to recover monoclonal antibodies. In Table 6, while almost 5× the hybrids were screened from CpG primed cells, no hybrids producing antibody specific to PCMA were found. In contrast, four clones producing specific antibody were flagged after screening just 98 hybrids. Further, all four hybridomas were of the stable IgG1 isotype.

In earlier examples, the novel adjuvant cocktails produced titers adequate for monoclonal antibody applications within a month from the first boost. This example with PCMA immunogen leaves no doubt that the inventive cocktails can produce antibodies (both monoclonal and polyclonal) to desired targets when traditional adjuvants are unable. Most significant, the CK19 antibody did not need purification prior to use as it was added to the inoculum in the whole ascites form. Antibody in the whole serum form, whole tissue culture supernatant form or semi-purified form is anticipated to work as well.

Using a Non-Specific Antibody as a Directing Molecule and a Saponin Stimulant to Enhance the Serum Response to Chlamydia Particles

Up to now, single genes or gene products have failed to match the protection achieved with whole chlamydia cells (R. C. Brunham et al., 2000. J. of Infectious Disease 538-543). More specifically, the majority of published work suggests only live chlamydia produce protective titers (J. J Donnelly, et al., 1997. Ann. Review of Immunology 15:617-648). While several labs are pursuing an attenuated chlamydia strain, the product from such efforts appears to be at least a decade away. As described in example 1 an inactivated chlamydia typically loses it's immunogenic characteristics. Example 1,5 and 6 show how the immunogenicity of an inactivated chlamydia particle can be restored by combining with directing molecule and stimulant class adjuvants.

The following example shows how inactivated chlamydia particles, when combined with stimulant and an antibody (not specific for immunogen) can produce substantial serum Ig titers.

### Example 5

| | |
|---|---|
| Immunogen | Chlamydia Trachomatis Infected MeCoy Cell, |
| | Lysate |
| Directing | Antibody (Murine, non-specific to immunogen |
| Molecule | |
| Stimulant | Saponin |

Immunogen Preparation

Approximately 36 ug of UV inactivated chlamydia trachomatis (LGV Type2) MeCoy cell lysate was dispensed into a one ml Nunc vial. Ninety micrograms of purified LI specific murine antibody was added in a 77 ul volume followed by 2 ul being 10 ug of purified saponin. The above was mixed and allowed to incubate at room temp for 10 minutes. Afterwards the volume was brought to 0.1 ml with HBSS allowing each of 5 animals to receive 200 ul of the prepared inoculum. The chlamydia was obtained from Microbix Biosystems Inc. 341 Bering Avenue Toronto Ontario Canada M8Z 3A8. The purified saponin was obtained from Superfos Specialty Chemical a/s Frydenlundsvej 30, DK-2950 Vedbaek, Denmark. The L1 specific antibody was generated in-house and affinity purified with a Protein-A resin before use. L1 is a cell adhesion molecule found in brain tissue. The L1 cell adhesion molecule was initially identified and characterized in mouse as a cell surface glycoprotein that mediates neuron-neuron and neuron-Schwann cell adhesion. An L1 transcript has been detected in neuroblastoma (IMR-32) and retinoblastoma (Y-79) cell lines. L1 is also expressed in the rhabdomyosarcoma cell lines RD and A-204 (R. A. Reid and J. J. Hemperly, 1992. J of Mol Neuroscience 3:127-135). Note the chlamydia used in this example was inactivated with UV radiation, wherein the prior chlamydia work (Example 1) chlamydia particles were inactivated by formalin. The chlamydia used in this example was not purified (existing as host cell lysate) and the L1 antibody was not specific for chlamydia.

Immunization

Five Balb/c mice were immunized with CFA and 5 received the novel adjuvant combination of stimulant and directing molecule. All animals received three initial IP immunizations spaced two weeks apart. Test bleeds were taken on day 39.

TABLE 7

| Control Group Balb/c Mice | | Test Group Balb/c Mice | |
|---|---|---|---|
| Chlamydia Immunogen + | | Chlamydia Immunogen + L1 Non-Specific | |
| CFA/IFA | | Antibody + Stimulant Quil-A Saponin | |
| Day 0 | IP Immunization | Day 0 | IP Immunization |
| Day 14 | IP Boost | Day 14 | IP Boost |
| Day 28 | IP Boost | Day 28 | IP Boost |
| Day 39 | Serum Sample | Day 39 | Serum Sample |

The test bleed taken after the third IP injection was titered vs. purified CT EBs in a solid phase ELISA.

Methods and Materials Used to Quantify Levels of Antibody to Chlamydia Immunogen

Materials: Nunc Maxisorb 96 well plates, LGV2 Chlamydia Trachomatis (cultured in human Hep2 (Cat #V501-D3271) obtained from East Coast Biologics, dehydrated milk powder, phosphate buffered saline (PBS), carbonate buffer, Tween-20, 5 ml glass tubes, test bleeds, TMB substrate Sigma T-8665, H₂SO₄, pool of goat anti-mouse IgG (1, 2A, 2B and 3) HRP conjugates from Accurate Chemical for titering serum samples

ELISA Procedure:

1. Coat NUNC Maxisorp plates with East Coast chlamydia particles at ˜1×10⁷ EBs per well for 1 hour at 37 degrees C.

2. Remove coating solution and block for 2 hours at 37 degrees C. with 250 ul/well 5% milk powder in PBS/Tween 20.

3 Remove block and rinse 3× with PBS-Tween 20

4. Add 100 ul of each serum sample diluted in 0.5-0.75% dehydrated milk powder and incubate for 1 hour at 37 degrees C.

5. Rinse 3× with PBS/Tween 20.

6. Add 100 ul of a Goat anti-mouse Ig HRP reagents diluted from 4-1 OK in PBS-Tween 20 and incubate at 37 degrees C. for 45 minutes.

7. Rinse 3× with PBS/Tween 20.

8. Add 100 ul of TMB substrate to each well and incubate for 30 minutes (not in direct light).

9. Stop the reaction with 100 ul of 0.5 molar sulfuric acid and read at 450 nm in a plate reader for record.

Sample Collection and Processing

All serum samples were obtained through orbital bleeds with a 100 ul micropipette from VWR Scientific Cat. 53432-921. The serum samples were allowed to clot overnight, followed by centrifugation to remove cells before freezing away. Assay dilutions ranged from 1:123 to 1:90K.

Serum Results

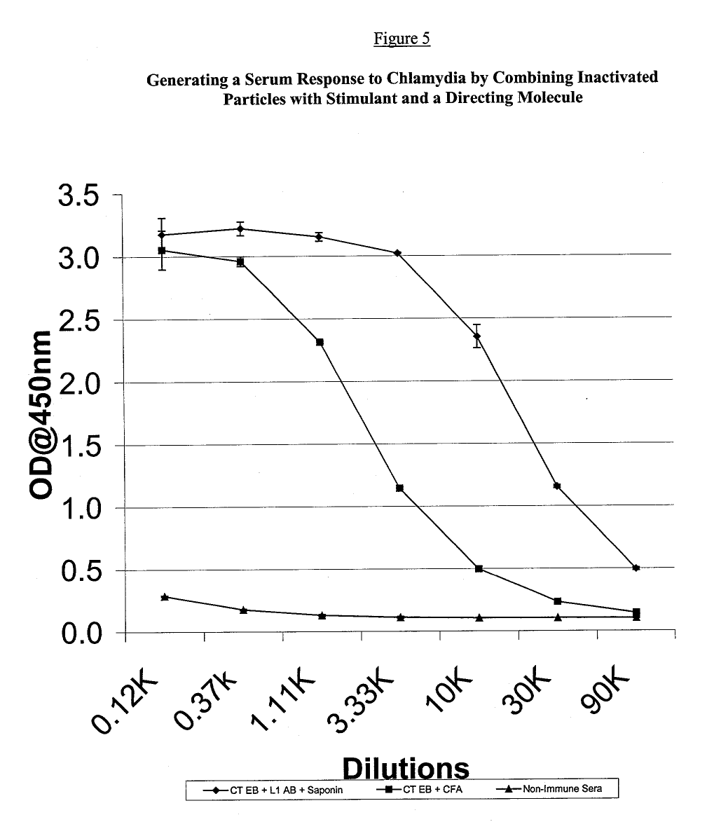

FIG. 5 shows a Freunds Adjuvant contributing to a serum titer of 10K and the adjuvant cocktail comprised of a non-specific antibody and saponin contributing to a 90K titer (>3× bkrd). The non-immune serum control was negative as expected.

The above results were obtained without any attempt at optimizing the immunogen dosage, neither ratio of adjuvants to immunogen, delivery route nor timing of sample collection.

Summary of Chlamydia Results

In a series of experiments one stimulant (purified saponin “Quil A”), when mixed with inactivated Chlamydia Trachomatis (CT) particles and a L1 non-specific antibody produced a serum titer that exceeded the titer raised with the same immunogen mixed with Freunds Adjuvant. The serum antibody enhancement was estimated at 9×. The results were obtained with a crude chlamydia prep demonstrating that an enhancement can be achieved without a purified immunogen. Note, when the chlamydia specific antibody described in Example 1 was combined with the UV inactivated particles (versus formalin treated) and saponin, then delivered IP, no enhancement was observed over the inoculum containing just chlamydia particles and saponin. Also when the inoculum above (UV Chlamydia-L1AB-Saponin) was delivered IM and ID, no improvement over inoculum containing just chlamydia particles and saponin was observed (data not shown). The last two results underscore the need to tailor cocktails per immunogen treatment and delivery route.

Using a Non-Specific Antibody as a Directing Molecule and a Saponin Stimulant to Generate Chlamydia Specific IgA in the Vaginal Mucosa

A key consideration when designing vaccines is to take into account the site of infection. Since chlamydia typically infects mucosal tissues, mucosal immunization may be required to achieve the best protection. Accordingly the following experiment was designed to determine if the novel adjuvants described in this invention show enhancements when delivered to the mucosa.

Without exception, the scientific literature indicates local (vaginal) anti-chlamydia Ig and specifically IgA coincides with the resolution of infection (R. P. Morrison et al., 1995. Infection and Immunity 4661-4668) and (A. L. Baron et al., 1984 Infection and Immunity 82-85). A mechanism for enhancing the local antibody response to chlamydia achieved with an inactivated chlamydia particle would provide great benefit to the general population and possibly curving the nearly 1 million new cases occurring each year in the U.S. Example 6 shows how intranasal delivery of inactivated chlamydia particles, when combined with stimulant and directing molecule adjuvants can produce vaginal mucosal IgA titers.

### Example 6

| | |
|---|---|
| Immunogen | Chlamydia Trachomatis Infected MeCoy Cell, |
| | Lysate |
| Directing | Antibody (Murine, non-specific to immunogen |
| Molecule | |
| Stimulant | Saponin |

Immunogen Preparation

Approximately 36 ug of UV inactivated chlamydia trachomatis (LGV Type2) MeCoy cell lysate was dispensed into an eppendorf tube. Forty-five micrograms of purified L1 specific murine antibody was added followed by 5 ug of purified saponin. The total volume was 35 ul and each animal received 7 ul of the prepared inoculum. The chlamydia lysate, saponin and L1 antibody were sourced as described earlier.

Immunization

Five Balb/c mice received just the chlamydia antigen, five received chlamydia antigen with saponin, five received chlamydia antigen with the L1 antibody and five received inoculum comprised of chlamydia, saponin and the L1 antibody. All animals received four immunizations over a two-month period followed by a vaginal wash two weeks later. Nasal immunizations were performed by applying ˜3.5 ul to the end of each nostril.

TABLE 8

| | Control Group | Control Group | Test Group |
|---|---|---|---|
| Control Group | Balb/c Mice | Balb/c Mice | Balb/c Mice |
| Balb/c Mice | Chlamydia | Chlamydia | Chlamydia |
| Chlamydia | Immunogen + | Immunogen + | Immunogen + L1 |
| Immunogen | Saponin | L1 Antibody | Antibody + Saponin |
| Day 0 | Day 0 | Day 0 | Day 0 |
| IN | IN | IN | IN |
| Immunization | Immunization | Immunization | Immunization |
| Day 14 | Day 14 | Day 14 | Day 14 |
| IN Boost | IN Boost | IN Boost | IN Boost |
| Day 33 | Day 33 | Day 33 | Day 33 |
| IN Boost | IN Boost | IN Boost | IN Boost |
| Day 64 | Day 64 | Day 64 | Day 64 |
| IN Boost | IN Boost | IN Boost | IN Boost |
| Day 85 Vaginal | Day 85 Vaginal | Day 85 Vaginal | Day 85 Vaginal |
| Fluid Sample | Fluid Sample | Fluid Sample | Fluid Sample |

Vaginal Sample Collection and Processing

Vaginal samples were obtained by washing the vault 2× with 25 ul of Hanks Buffered Saline. To the 50 ul sample, 12.5 ul of aprotinin at 20 ug/ml and 12.5 ul of 0.01M DTT were added. The vaginal samples were vortexed and frozen immediately. On the day of assay, samples were thawed and centrifuged at 10K rpm for clarification.

Methods and Materials Used to Quantify Levels of Chlamydia Specific IgA

The vaginal fluid samples were titered vs. purified CT EBs as described for Example 5 with only the following exceptions: A single screening dilution of 1:8 was used for vaginal fluid samples and the secondary conjugate was changed to Goat anti-Mouse IgA HRP cat 1040-05 from Southern Biotech. Dilutions were made in diluent containing 0.5% dehydrated milk powder and 10% media being 20% serum.

Vaginal Fluid Results

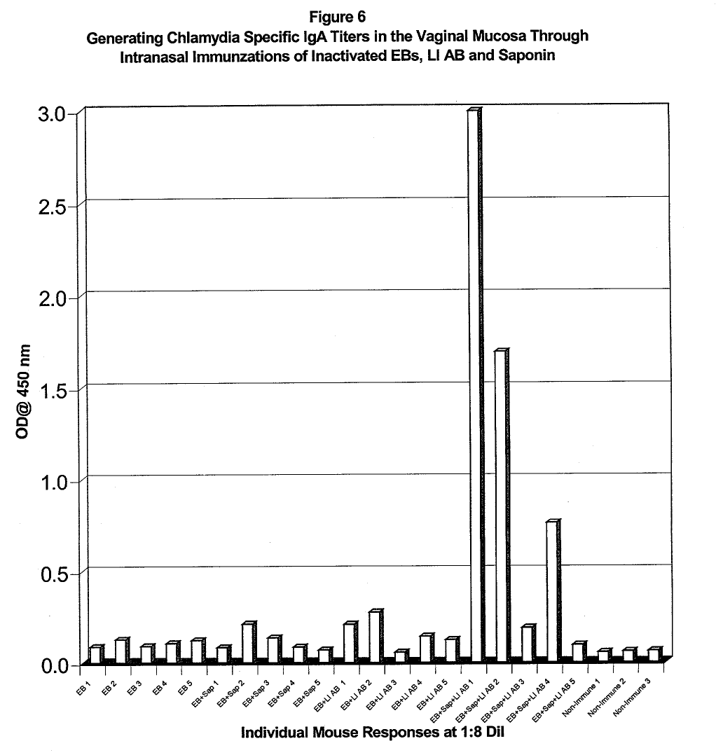

FIG. 6 shows individual responses for the 5 groups of mice. Mucosal sample screens showed ⅗ animals immunized with the infected cell lysate—L1 Antibody—Saponin produced chlamydia specific IgA. Inoculums comprised of immunogen, immunogen mixed with saponin and immunogen mixed with the L1 antibody failed to generate positive signals at the 1:8 screening dilution (>2× bkgd and >0.3 OD). The three non-immune samples served as background.

Summary of Chlamydia Results

In a series of intranasal delivery experiments only one inoculum, comprised of a stimulant` (purified saponin “Quil A”), an inactivated Chlamydia Trachomatis infected cell lysate and a L1 antibody (not specific for immunogen) produced vaginal IgA titers. The results were obtained with a crude chlamydia prep demonstrating that an enhancement can be achieved without purified immunogen. The above results were obtained without any attempt at optimizing the immunogen dosage, neither ratio of adjuvants to immunogen, or timing of sample collection or immunization interval. Note the adjuvant cocktail that enabled an IgA response in the mucosa was first flagged in Example 5, an experiment where the inoculums were given IP. Examples 5 and 6 provide evidence that such adjuvant cocktails intended for mucosal applications can be screened in the peritoneum whereby responses are measured in serum and with better precision.

Using Alpha 2-Macroglobulin as a Directing Molecule and a Saponin Stimulant to Generate Chlamydia Specific IgA in the Vaginal Mucosa

In Example 7, a formulation consisting of prelyophilized murine alpha 2-M, saponin and an inactivated prelyophilized chlamydia particle were delivered nasally. The formulation produced vaginal IgA levels similar to those achieved with an inoculum-containing antibody.

### Example 7

| | |
|---|---|
| Immunogen | Chlamydia Trachomatis Purified EBs |
| Directing | Murine alpha 2-macro globulin or |
| Molecule | Murine antibody to L1 |
| Stimulant | Saponin |

Immunogen Preparation

Dispensed into an eppendorf tube was approximately 8×10⁸ of UV-psoralen inactivated chlamydia trachomatis (LGV Type2), 20 ug of purified saponin and 115 ug of purified L1 murine antibody or 10 ug of murine alpha 2-macroglobulin. The total volume was 50 ul and each animal received 10 ul of the prepared inoculum. The saponin and L1 antibody were sourced as described earlier. The chlamydia EBs were obtained from East Coast Biologics and are equivalent to the material used in the ELISA in Examples 5,6, and 7. The murine alpha 2-macroglobulin was prepared in-house by passing whole serum collected from Balb/c mice over an affinity resin with immobilized rabbit anti mouse alpha 2-macroglobulin. The chlamydia particles, alpha 2-M, L1 antibody and saponin were previously lyophilized and hydrated just prior to preparing inoculum. The alpha 2-M was not conjugated to the immunogen as was done in examples 2 and 3.

Immunization

Five Balb/c mice received inoculum comprised of chlamydia, saponin and the L1 antibody. A second group of five received inoculum comprised of chlamydia, saponin and alpha 2-macroglobulin. Both groups received three intranasal immunizations over one month followed by a vaginal wash eight days later. Nasal immunizations were performed by applying ˜5 ul to the end of each nostril.

TABLE 9

| Control Group 5 Balb/c Mice | | Test Group 5 Balb/c Mice | |
|---|---|---|---|
| Chlamydia Immunogen + L1 | | Chlamydia Immunogen + | |
| Antibody + Saponin | | Alpha 2-M + Saponin | |
| Day 0 | IN Immunization | Day 0 | IN Immunization |
| Day 15 | IN Boost | Day 15 | IN Boost |
| Day 28 | IN Boost | Day 28 | IN Boost |
| Day 36 | Vaginal Fluid Sample | Day 36 | Vaginal Fluid Sample |

The vaginal fluid samples were collected, processed, diluted and titered vs. purified CT EBs as described in example 6.

Vaginal Fluid Results

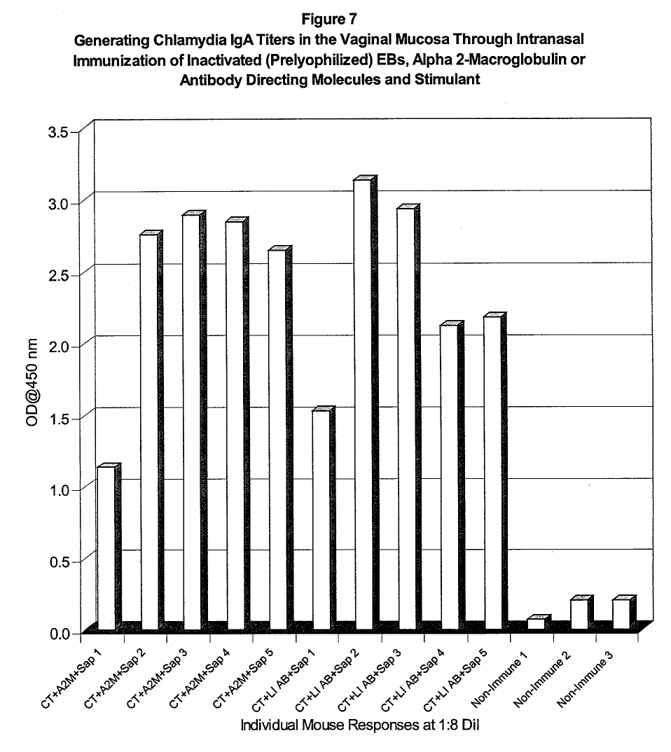

FIG. 7 displays individual responses for the 2 groups of mice. Each bar was an average of two ELISA wells. Five of five animals immunized with the inoculum containing L1 produced chlamydia specific IgA. Five of five animals immunized with the inoculum containing alpha 2-M also produced chlamydia specific IgA. Samples were again diluted 1:8 for screening and samples called positive when signals >2× bkgd and ≧0.3 OD were observed. As indicated by the signal intensity, inoculum-containing alpha 2-M produced titers competitive to those generated by the control group. The three non-immune samples served as background.

Summary of Chlamydia Results

In the prior Example 6, Chlamydia antigen delivered nasally with saponin and L1antibody led to vaginal IgA titers. Example 7 demonstrates alpha 2-M can substitute for antibody. The results were obtained with a lyophilized and inactivated chlamydia prep making these formulations more conducive with commercialization.

The data within shows six distinct combinations of stimulant and directing molecule combinations delivered by various routes leading to additive and synergistic improvements to serum antibody titer without signs of toxicity.

In summary, the examples provided show an unexpected synergistic enhancement when stimulants and directing molecules are combined. The examples include five diverse immunogens, four different directing molecules, two unique stimulants, six adjuvant pairs (all 7 examples were comprised of directing molecule and stimulant) and three separate routes. The positive interaction between these stimulants and directing molecules suggests a broad predictable mechanism is involved and should be easily exploited for other immunogens. Further, any directing molecule (having receptor on APC) should produce similar enhancements.

While the examples shared were subcutaneous, intraperitoneal and intranasal; similar enhancements have been observed by vaginal, intramuscular, intradermal (data not shown), intravenous, topical, intravascular and the other mucosal such as oral and rectal routes are anticipated to be suitable for the practice of this invention. Success has also been obtained with virus particles and disrupted virions (data not shown).

Having safe cocktails comprised of stimulants and directing molecules may lead to improvements in vaccination by routes where depots such as alum are excluded. These new cocktails will likely be more compatible with new micro needle and micro abraders devices where alum and other large particles will find restrictions.

The prior work in other labs showed combinations of depots with directing molecules or depots with stimulants leading to mere “additive enhancements” over their constituents. Herein, combining directing molecules and stimulants leads to synergistic enhancements as demonstrated in Example 1.

In Example 2, the combination of a directing molecule with a stimulant allowed for the generation of functional titers without toxicity. With a synergistic enhancement in titer, the concentration of stimulant can be reduced to safe levels where any indications of local or systemic toxicity disappear.

Finally, advocates for the independent use of directing molecules or certain stimulants claim their reagents require less immunogen. However, Example 3 clearly demonstrates that commercially available alpha 2-M and CpG DNA used alone, fail to elicit functional titers with 40 ng of immunogen. In contrast, the combination produced titers adequate for monoclonal antibody applications within a month from the first boost. This new ability to work with ultra low quantities of immunogen improves the prospect of generating antibodies to clinical targets present only in small amounts, such as many cancer markers.

Success with C. trachomatis by numerous routes provides a clear strategy for related organisms such as C. pneumoniae and psittaci. The transmucosal results obtained by administering chlamydia nasally and obtaining IgA in the vaginal vault are particularly valuable, since nasal immunization requires less skill and the disposable does not involve a needle. The success in the peritoneum makes malignances of the peritoneal cavity prime candidates for the inventive adjuvant cocktails described within.

Immediately, antibody houses and academic labs can do away with Complete Freunds Adjuvant and other toxic substances typically used to generate research and commercial grade antibody reagents.

For example, when generating monoclonal antibody reagents, the practitioner may want to select an adjuvant cocktail and route that primes the spleen, the largest single source of B-cells in the body. If the goal is to generate protection against a pathogen of the lower mucosa, then the practitioner may want to select for adjuvant cocktails that can produce vaginal IgA titers when delivered nasally (transmucosal).

This invention will teach immunologists to expect each stimulant and directing molecule to work with varying efficiency depending on the immunogen, immunogen treatment, delivery route and the type of immune response pursued. However the best performing adjuvants will always possess the traits of a stimulant and directing molecule. This invention places commercially available materials into helpful categories based on function and teaches how to pair candidates from each category for the best possible outcome. The examples within provide details as to the level of immunogen purity and directing molecule purity needed to realize results. The concentrations for several types of immunogens, directing molecules and stimulants are provided along with suggested ratios to achieve the desired immune response.

After reviewing the examples within this invention those skilled in the art will appreciate the magnitude of the enhancements and will also understand that a single pair of directing molecule/stimulant type adjuvants may not deliver the best performance for all immunogens and applications.

Any conventional method for preparing immunogen or vaccine compositions may be employed in preparing the composition. It may be desirable to prepare a complex of the immunogen and directing molecule to enhance the beneficial effects of the composition.

It is expected that the vaccine or inoculum will lead to improved protection and/or treatments. Further, the use of the composition enhances the discovery frequency and quality of antibodies intended for diagnostics and therapy as generated by established monoclonal, polyclonal and recombinant techniques without the toxic side effects to man and animal.

It's expected that adjuvants possessing both stimulant and directing properties will enhance topical delivery as well. As Mitragotri et al., reported that molecules within the size-range of the adjuvants described within could be coaxed into crossing the stratum corneum using low frequency ultrasound (Mitragori et al. 1995 Science 269:850-853).

What is claimed is:

1. An immunogenic composition comprising an immunogen, a first adjuvant functioning as a directing molecule and a second adjuvant functioning as a stimulant, wherein the first and second adjuvants are chemically distinct molecules and the immunogen and first and second adjuvants are present in amounts sufficient to result in an improved immune response relative to that resulting from the immunogen and just one of the first or second adjuvants.

2. The immunogenic composition of claim 1 wherein the stimulant is a weak stimulant.

3. The immunogenic composition of claim 1 free from agents causing visible external toxic or allergic symptoms.

4. The immunogenic composition of claim 1 suitable for intranasal, intraperitoneal, or subcutaneous delivery.

5. The immunogenic composition of claim 1 suitable for intradermal, intramuscular, intravenous, intravascular, vaginal, rectal, oral or topical delivery.

6. The immunogenic composition of claim 1 that is delivered to the mucosa

7. The immunogenic composition of claim 1 wherein the immune response is measured by tissue or serum Ig

8. The immunogenic composition of claim 1 wherein the stimulant is saponin or derivatives thereof.

9. The immunogenic composition of claim 4 wherein the saponin derivative is a saponin component.

10. The immunogenic composition of claim 1 wherein the saponin is synthetic

11. The immunogenic composition of claim 1 wherein the stimulant is CpG DNA.

12. The immunogenic composition of claim 1 wherein the stimulant is nucleic acid.

13. The immunogenic composition of claim 1 wherein the stimulant is a substance endogenous to humans.

14. The immunogenic composition of claim 1 wherein the stimulant is a substance exogenous to humans.

15. The immunogenic composition of claim 1 wherein the stimulant is a cytokine.

16. The immunogenic composition of claim 1 wherein the stimulant is a chemokine.

17. The immunogenic composition of claim 1 wherein the stimulant is contributes to APC migration.

18. The immunogenic composition of claim 1 wherein the stimulant contributes to APC maturation.

19. The immunogenic composition of claim 1 wherein the stimulant attracts APC.

20. The immunogenic composition of claim 1 wherein the immunogen is an antigen.

21. The immunogenic composition of claim 20 wherein the antigen is encoded by nucleic acid.

22. The immunogenic composition of claim 20 wherein the antigen is whole cell, protein, or protein mixture.

23. The immunogenic composition of claim 22 wherein the protein mixture is a membrane extract.

24. The immunogenic composition of claim 23 wherein the membrane extract originates from mammalian cells.

25. The immunogenic composition of claim 22 wherein the whole cell is virus or bacteria.

26. The immunogenic composition of claim 25 wherein the bacteria is chiamydia.

27. The immunogenic composition of claim 26 wherein the chlamydia is trachomatis, pneumonia or psittaci.

28. The immunogenic composition of claim 26 wherein the chlamydia is an elementary body.

29. The immunogenic composition of claim 28 wherein the chlamydia elementary body is inactivated with UV light.

30. The immunogenic composition of claim 28 wherein the chlamydia elementary body is inactivated with formalin.

31. The immunogenic composition of claim 28 wherein the chlamydia elementary body is inactivated by psoralen.

32. The immunogenic composition of claim 1 wherein the directing molecule is alpha 2-macroglobulin (Alpha 2-M) or CD91 binding fragment of A2M.

33. The immunogenic composition of claim 1 wherein the directing molecule is an antibody or an immunogen specific fragment thereof.

34. The immunogenic composition of claim 33 wherein the antibody or its fragment is specific for an antigen presenting cell (APC) receptor.

35. The immunogenic composition of claim 33 wherein the antibody or its fragment possesses an epitope capable of binding to an APC receptor.

36. The immunogenic composition of claim 33 wherein antibody or immunogen specific fragment is not specific for the immunogen.

37. The immunogenic composition of claim 33 wherein the antibody complementarity determining regions (CDRs) are specific for the APC.

38. The immunogenic composition of claim 33 wherein the antibody CDRs are not specific for the APC.

39. The immunogenic composition of claim 33 wherein the antibody CDRs are specific for the immunogen.

40. The immunogenic composition of claim 33 wherein the antibody CDRs are not specific for the immunogen.

41. The immunogenic composition of claim 33 wherein the antibody CDRs are specific for the stimulant.

42. The immunogenic composition of claim 33 wherein the antibody CDRs are not specific for the stimulant.

43. The immunogenic composition of claim 1 wherein the directing molecule binds to transferrin, manose, asialoglycoproteins receptor or CD91.

44. The immunogenic composition of claim 1 wherein the directing molecule is a complement or heat shock protein or fragment thereof capable of binding to APC.

45. The immunogenic composition of claim 1 where the directing molecule is an opsonizing molecule.

46. The immunogenic composition of claim 1 wherein the directing molecule comprises a molecule directly or indirectly linked to an APC receptor.

47. The immunogenic composition of claim 1 wherein the directing molecule and the immunogen form a complex.

48. The immunogenic composition of claim 1 wherein the composition is a vaccine.

49. The immunogenic composition of claim 1 wherein the composition is frozen, lyophilized, freeze dried or otherwise reconstitutable.

50. The immunogenic composition of claim 1 which is substantially liposome-free.

51. The immunogenic composition of claim 1 which is substantially free of alum.

52. The immunogenic composition of claim 1 further comprising at least one antibody or fragment thereof which is either specific or not specific for the immunogen.

53. A method for inducing an immune response by administering an immunogenic composition to a subject at a desired site, wherein the immunogenic composition comprises:
an immunogen, a first adjuvant functioning as a directing molecule and a second adjuvant functioning as a stimulant, wherein the first and second adjuvants are chemically distinct molecules and the immunogen and first and second adjuvants are present in amounts sufficient to result in an improved immune response relative to that resulting from the immunogen and just one of the first or second adjuvants.

54. The method of claim 53 wherein the administration is by intranasal, intraperitoneal, or subcutaneous delivery.

55. The method of claim 53 wherein the administration is by intradermal, intramuscular, intravenous, intravascular, vaginal, rectal, oral or topical delivery.

56. The method of claim 53 wherein the administration is by mucosal delivery.

57. The method claim 53 wherein the immune response involves antibody formation.

58. The method claim 57 wherein the antibody formation is transmucosal.

59. The method accordingly to claim 57 wherein the antibody has Ka values between 10⁴-10¹³ moles/liter.

60. The method accordingly to claim 57 wherein the antibody is in the form of unpurified whole ascites.

61. The method accordingly to claim 57 wherein the antibody is in the form of unpurified whole serum.

62. The method accordingly to claim 57 wherein the antibody is in the form of a whole cell culture supernatant.

63. The method accordingly to claim 57 wherein the antibody is-a semi-purified form.

64. The method accordingly to claim 57 further comprising the step of recovering the antibody from the subject.

65. The method according to claim 57 further comprising the step of analyzing the affinity(s) of the recovered antibody.

66. The method of claim 53 wherein the method is immunotherapy.

67. The method accordingly to claim 53 wherein the immune response is protective.

68. The method according to claim 53 wherein the administering step involves mucosal administration.

69. The method according to claim 53 wherein the administering step involves administration by more than one route.

70. The method according to claim 53 wherein the administering step involves administration by more than one route either simultaneously or in sequence.

71. The method according to claim 53 wherein the administering is a series of vaccinations.

72. A method for preparing the composition of claim 1 comprising mixing immunogen, directing molecule, and stimulant.

73. The method of claim 72 wherein the immunogen and directing molecule are complexed and then mixed with the stimulant.

74. The immunogenic composition of claim 1 wherein the stimulant is selected from the group consisting of; GM-CSF, IL-1-beta, IL-2, IL-4, IL-7, IL-12, monophosphoryl lipid A (MPL), 3-Q-desacyl-4′-monophosphoryl lipid A (3D-MLA), IL-1beta 163-171 peptide (Sclavo Peptide), 25-dihydroxyvitamin D3, calcitinin-gene regulated peptides, Dehydroepiandrosterone (DHEA), N-Acetylglucosaminyl-(P1-4)-N-acetylmuramyl-L-alanyl-D-glutamine (GMDP), dimethyl dioctadecyla or disteary ammonium bromide (DDA)/Zinc L-proline, muramyl dipeptide (MDP), N-Acetylglucosaminyl-(P1-4)-N-acetylmuramyl-L-alanyl-D-glutamine (GMDP), N-acetyl muramyl-L-threonyl-D-isoglutamine (Threonyl-MDP), N-acetyl-L-alanyl-D-isoglutaminyl-L-alanine-2-(1,2-dipalmitoyl-sn-glycero-3-(hydroxy-phosphoryloxy) ethylamide monosodium salt (MTP-PE), Nac-Mur-L-Ala-D-Gln-OCH3, Nac-Mur-L-Thr-D-isoGln-sn-glycerol dipalmitoyl, Nac-Mur-D-Ala-D-isoGln-sn-glycerol dipalmitoyl, 1-(2-methypropyl)-IH-imidazo[4,5-c]quinolin-4-amine, 4-Amino-otec-dimethyl-2-ethoxymethyl-1H-imidazo[4,5-c]quinoline-1-ethanol, N-acetylglucosaminyl-N-acetylmuramyl-L-Ala-D-isoGlu-L-Ala-glycerol dipalmitate (DTP-GDP), N-acetylglucosaminyl-N-acetylinuramyl-L-Ala-D-isoGlu-L-Ala-dipalmitoxy propylamide (DTP-DPP), gamma interferon, 7-allyl-8-oxoguanosine, poly-adenylic acid-poly-uridylic acid complex, MIP-1a, MIP-3a, dibutyl phthalate, dibutyl phthalate analogues and C5a.
